(19) United States
(12) Patent Application Publication
(10) Pub. No.: US 2026/0134987 A1
(43) Pub. Date: May 14, 2026

(54) ORTHOPAEDIC SURGICAL SYSTEM IMAGE CAPTURE

(71) Applicant: DePuy Ireland Unlimited Company, Ringaskiddy (IE)

(72) Inventors: Andrew M. Hudson, Warsaw, IN (US); Christopher Hunt, Leeds (GB); Clinton A. Beck, Fort Wayne, IN (US); Steven Coker, II, Fort Wayne, IN (US); Taylor Wisler, Warsaw, IN (US)

(21) Appl. No.: 19/327,217

(22) Filed: Sep. 12, 2025

Related U.S. Application Data
(60) Provisional application No. 63/718,198, filed on Nov. 8, 2024.

(51) Int. Cl. G16H 40/63; A61B 34/10; A61B 90/98; G06T 5/80; G06T 7/00; G06T 7/73; G16H 10/60; H04N 7/18

(52) U.S. Cl. CPC G16H 40/63; A61B 34/10; A61B 90/98; G06T 5/80; G06T 7/0014; G06T 7/74; G16H 10/60; H04N 7/181; A61B 2034/105; G06T 2207/10116; G06T 2207/30008; G06T 2207/30168; G06T 2207/30204

(57) ABSTRACT

A method of operating an orthopaedic surgical system may include displaying a unique identifier on a visual display of a first device positioned in an operating room, the unique identifier being associated with a digital medical record of a patient positioned in the operating room, capturing the unique identifier with a camera of a second device positioned in the operating room, establishing a unique electronic connection between the second device and the digital medical record of the patient based on the unique identifier, capturing one or more images of the patient with the camera of the second device, transferring the one or more images from the second device to the digital medical record of the patient using the unique electronic connection, and accessing the one or more images with the first device to guide performance of an orthopaedic surgical procedure on the patient.

This application claims the benefit of U.S. Provisional Patent Application Ser. No. 63/718,198, filed Nov. 8, 2024, the entirety of which is incorporated herein by reference.

### TECHNICAL FIELD

The present disclosure relates generally to computer-assisted surgery systems for use in orthopaedic surgical procedures and, more particularly, to technologies for image capture in such systems.

### BACKGROUND

Joint arthroplasty is a well-known surgical procedure by which a diseased and/or damaged natural joint is replaced by a prosthetic joint. An orthopaedic surgeon may use an orthopaedic surgical system to assist with planning and/or performing the joint arthroplasty, such as guiding placement of prosthetic joint components relative to the patient's bones. To do so, the orthopaedic surgical system may obtain and analyze one or more images of the patient's bones. For example, preoperative and/or intraoperative images may be used in reconstructive orthopedics to analyze biomechanics. Typically, the images of the patient's bones must be transferred from an imaging device used to initially capture the images to another part of the orthopaedic surgical system for analysis.

The initial configuration and ongoing support of orthopaedic surgical systems that incorporate image transmission functionality presents challenges in medical environments, including the reliance on IT (Information Technology) and engineering support to network and/or program systems to support direct image transfer. When portable orthopaedic surgical systems are brought into a hospital or surgery center for short-term use, such as for a specific surgery, setting up networked systems to facilitate image transmission can present an even greater challenge due to both technology and compliance protocols. Barriers to networking systems often impede the use of systems that rely upon the direct transfer of medical images.

Healthcare professionals sometimes share information informally by taking camera images of original medical images captured by imaging devices. However, taking a camera image in lieu of directly transferring the original medical image is not traditionally supported in clinical systems, due to concerns regarding security, privacy, and the potential to introduce image distortion, among others.

### SUMMARY

According to one aspect of the present disclosure, a method of operating an orthopaedic surgical system may comprise displaying a unique identifier on a visual display of a first device positioned in an operating room, the unique identifier being associated with a digital medical record of a patient positioned in the operating room, capturing the unique identifier with a camera of a second device positioned in the operating room, establishing a unique electronic connection between the second device and the digital medical record of the patient based on the unique identifier, capturing one or more images of the patient with the camera of the second device, transferring the one or more images from the second device to the digital medical record of the patient using the unique electronic connection, and accessing the one or more images with the first device to guide performance of an orthopaedic surgical procedure on the patient.

In some embodiments, transferring the one or more images from the second device to the digital medical record of the patient using the unique electronic connection may comprise wirelessly transmitting the one or more images.

In some embodiments, accessing the one or more images with the first device to guide performance of the orthopaedic surgical procedure may comprise generating a surgical plan for the orthopaedic surgical procedure with the first device using the one or more images.

In some embodiments, the digital medical record of the patient may be stored on a server positioned outside of the operating room, and accessing the one or more images with the first device may comprise retrieving the one or more images from the digital medical record over a network connecting the first device and the server.

In some embodiments, the method may further comprise generating, with the server, a unique token associated with the digital medical record of the patient, transmitting the unique token from the server to the first device, and generating, with the first device, the unique identifier using the unique token.

In some embodiments, establishing the unique electronic connection between the second device and the digital medical record of the patient may comprise extracting, with the second device, the unique token from the unique identifier and transmitting the unique token from the second device to the server to establish the unique electronic connection.

In some embodiments, the unique identifier may be a Quick Response (QR) code.

In some embodiments, the method may further comprise capturing one or more initial images of the patient with a third device that is not connected to the second device. Capturing the one or more images of the patient with the second device may comprise capturing, with the camera of the second device, one or more images of a visual display of the third device while the visual display of the third device displays the one or more initial images.

In some embodiments, capturing the one or more initial images of the patient with the third device may comprise capturing one or more X-ray images showing one or more bones of the patient.

In some embodiments, displaying the unique identifier on the visual display of the first device may be performed in response to a failure of an attempted transfer of the one or more initial images of the patient from the third device to the digital medical record.

In some embodiments, the visual display of the third device may include one or more physical markers having a predefined pattern, and the one or more images of the visual display of the third device captured by the second device may comprise the one or more initial images of the patient and the one or more physical markers.

In some embodiments, the predefined pattern of the one or more physical markers may comprise at least three identifiable locations spaced apart from one another at predefined distances.

In some embodiments, the one or more physical markers may comprise a single physical marker including the at least three identifiable locations.

In some embodiments, the one or more physical markers may comprise three physical markers each including one of the at least three identifiable locations.

In some embodiments, each physical marker of the one or more physical markers may comprise a decal applied to the visual display of the third device.

In some embodiments, each decal may be configured to destroy at least a portion of the predefined pattern if the decal is removed from the visual display of the third device.

In some embodiments, the second device may confirm that the predefined pattern of the one or more physical markers in the one or more images of the visual display of the third device captured by the second device matches an expected pattern uniquely associated with the third device prior to transferring the one or more images to the digital medical record of the patient using the unique electronic connection.

In some embodiments, the method may further comprise detecting distortion in the one or more images captured by the second device by determining that the at least three identifiable locations are not spaced apart from one another at the predefined distances in the one or more images.

In some embodiments, the method may further comprise manipulating the one or more images captured by the second device to correct the distortion.

In some embodiments, the method may further comprise accounting for the distortion in the one or more images when guiding the performance of the orthopaedic surgical procedure on the patient.

In some embodiments, the predefined pattern of the one or more physical markers may comprise a Quick Response (QR) code.

In some embodiments, the predefined pattern of the one or more physical markers may comprise a scaling strip.

According to another aspect, a method of operating an orthopaedic surgical system may comprise capturing an X-ray image showing one or more bones of a patient using an imaging device positioned in an operating room, displaying the X-ray image on a visual display associated with the imaging device, wherein the visual display includes one or more physical markers having a predefined pattern, capturing a digital image of the visual display with a camera positioned in the operating room, wherein the digital image comprises the one or more physical markers and the X-ray image displayed on the visual display, and comparing a pattern of the one or more physical markers shown in the second image to the predefined pattern expected for the one or more physical markers to identify distortion in the digital image.

In some embodiments, the predefined pattern of the one or more physical markers may comprise at least three identifiable locations spaced apart from one another at predefined distances.

In some embodiments, the one or more physical markers may comprise a single physical marker including the at least three identifiable locations.

In some embodiments, the one or more physical markers may comprise three physical markers each including one of the at least three identifiable locations.

In some embodiments, comparing the pattern of the one or more physical markers shown in the digital image to the predefined pattern expected for the one or more physical markers may comprise determining that the at least three identifiable locations are not spaced apart from one another at the predefined distances to identify distortion in the digital image.

In some embodiments, the method may further comprise manipulating at least a portion of the digital image corresponding to the X-ray image to correct the distortion.

In some embodiments, the method may further comprise accounting for the distortion when calculating a distance or an angle between anatomical features shown in a portion of the digital image corresponding to the X-ray image.

In some embodiments, the predefined pattern of the one or more physical markers may comprise a Quick Response (QR) code.

In some embodiments, the predefined pattern of the one or more physical markers may comprise a scaling strip.

In some embodiments, each physical marker of the one or more physical markers may comprise a decal applied to the visual display associated with the imaging device.

In some embodiments, each decal may be configured to destroy at least a portion of the predefined pattern if the decal is removed from the visual display associated with the imaging device.

In some embodiments, the method may further comprise blocking transmission or further use of the second image if the pattern of the one or more physical markers shown in the second image does not match to the predefined pattern expected for the one or more physical markers.

According to yet another aspect, the present disclosure includes embodiments of computer-readable media storing a plurality of instructions that, when executed by one or more processors, cause the one or more processors to perform the steps of any of the methods described herein.

According to still another aspect, the present disclosure includes embodiments of orthopaedic surgical systems configured to perform the steps of any of the methods described herein.

### BRIEF DESCRIPTION OF THE DRAWINGS

The detailed description particularly refers to the following figures, in which:

FIG. 1 is simplified diagram of an orthopaedic surgical system;

FIGS. 2A-2D are a simplified flowchart of a method that may be performed using the orthopaedic surgical system of FIG. 1; and

FIGS. 3A-3D are illustrative images captured using the orthopaedic surgical system of FIG. 1 and the method of FIGS. 2A-2D.

### DETAILED DESCRIPTION OF THE DRAWINGS

While the concepts of the present disclosure are susceptible to various modifications and alternative forms, specific illustrative embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

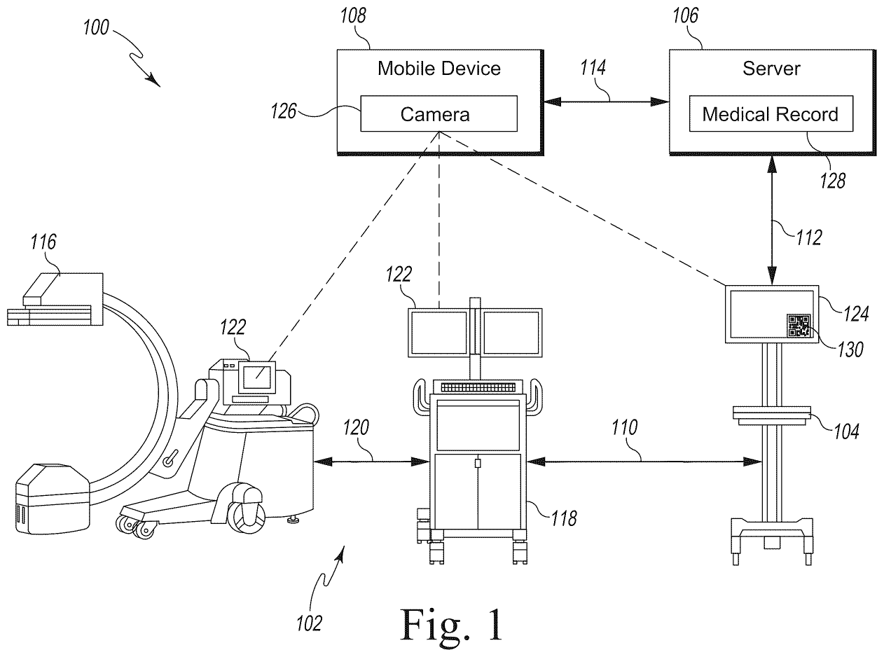

Referring now to FIG. 1, one embodiment of an orthopaedic surgical system 100 according to the present disclosure is shown. The orthopaedic surgical system 100 illustratively includes an imaging device 102, a local device 104, a server 106, and a mobile device 108. As described further below, the imaging device 102 may communicate with the local device 104 via a communication link 110, the local device 104 may communicate with the server 106 via a communication link 112, and the server 106 may communicate with the mobile device 108 via a communication link 118. It is contemplated that some embodiments of the presently disclosed orthopaedic surgical system 100 may omit one or more of the foregoing components and/or communication links. For instance, in some embodiments, the direct link 110 between the imaging device 102 and the local device 104 may be omitted. As another example, in some embodiments, the server 106 may be omitted and the mobile device 108 and the local device 104 may communicate directly with one another (rather than via the server 106).

During at least part of an orthopaedic surgical procedure on a patient, the imaging device 102, the local device 104, and the mobile device 108 are each positioned in the same operating room as the patient (though the imaging device 102, the local device 104, and the mobile device 108 need not be present in the operating room for the entire orthopaedic surgical procedure or at other times). When used, however, the server 106 is typically not present in the operating room. Instead, the server 106 may be located in another part of the hospital or surgery center, or in another building altogether.

The imaging device 102 may be embodied as any type of device or collection of devices capable of generating medical images of the bony anatomy of the patient. In the illustrative embodiment, the imaging device 102 includes a C-arm X-Ray imaging machine 116 capable of generating two-dimensional medical images. The illustrative imaging device 102 also includes a display console 118 coupled to the C-arm 116 by a communication link 120. In use, X-ray images 304 captured by the C-arm 116 are transferred via the link 120 to the display console 118 where the X-ray images 304 are displayed on a visual display 122 of the display console 118. Additionally or alternatively, the X-ray images may be displayed on a visual display 118 of the C-arm 116. In other embodiments, the functionality of the C-arm 116 and the display console 118 may be combined into a single imaging device 102. It is also contemplated that, in other embodiments, the imaging device 102 may be embodied as (or include) an imaging device capable of generating three-dimensional medical images, such as a computed tomography (CT) or magnetic resonance imaging (MRI) machine. In such embodiments, the medical images displayed on the visual display 122 of the imaging device 102 may be three-dimensional images, two-dimensional slices of the three-dimensional medical images, or simulated two-dimensional medical images generated by projecting the three-dimensional medical images onto a two-dimensional plane.

The local device 104 is illustratively embodied as computer hardware and associated software carried by a movable cart. The local device 104 may be embodied as any type of computer or computation device capable of performing the functions described herein. For example, the local device 104 may be embodied as a desktop computer, a surgical navigation computer, a laptop computer, a tablet computer, a smartphone, a mobile computer, a smart device, a wearable computer system, or other computer or computer device. The local device 104 may include one or more processors, memory, input/output (“I/O”) subsystems, data storages, communication systems, and peripheral devices, including at least one visual display 124. The visual display 124 may be embodied as any type of display capable of displaying information to a user (e.g., the orthopaedic surgeon) of local device 104. For example, the display 124 may be embodied as a liquid crystal display (LCD), a light emitting diode (LED) display, an organic light emitting diode (OLED), a cathode ray tube (CRT) display, a plasma display, an augmented or virtual reality headset, and/or other display device. In some embodiments, the display 124 may include a touchscreen, which may be configured to receive input from the orthopaedic surgeon based on a tactile interaction. Additionally, in some embodiments, the display 124 may be separate from other portions of the local device 104, but communicatively coupled thereto. Of course, the local device 104 may include additional or other components, such as those commonly found in a typical computer device, in other embodiments. Additionally, in some embodiments, one or more of the illustrative components may be incorporated in, or otherwise form a portion of, another component.

The server 106 may be embodied as any type of computer or computation device capable of performing the functions described herein. For example, the server 106 may be embodied as a rack-mounted computer, a network appliance, a desktop computer, a laptop computer, a tablet computer, or other computer or computer device. Similar to the local device 104, the server 106 may include one or more processors, memory, input/output (“I/O”) subsystems, data storages, communication systems, and peripheral devices. The local device 104 and the server 106 both execute software and communicate with one another to perform surgical planning at the direction of the orthopaedic surgeon. In embodiments of the system 100 that do not include the server 106, the local device 104 may execute standalone software to perform surgical planning at the direction of the orthopaedic surgeon.

As shown in FIG. 1, the server 106 may store digital medical records for the orthopaedic surgeon's patients, including a digital medical record 128 of the patient positioned in the operating room (on whom the orthopaedic surgeon is performing the orthopaedic surgical procedure). The digital medical record 128 may include a number of medical images (such as those described above in conjunction with the imaging device 102), a surgical plan for the orthopaedic surgical procedure (further described below), and other patient-specific electronic data. In some embodiments, part or all of the digital medical record 128 may be stored on the local device 104 (either independent of or duplicative of portions of the digital medical record 128 stored on the server 106). For instance, in embodiments of the system 100 that do not include the server 106, the entire digital medical record 128 for the patient may be maintained on the local device 104.

In the illustrative embodiment, the system 100 further includes at least one mobile device 108. The mobile device(s) 108 may be embodied as any type of computer or computation device capable of performing the functions described herein. For example, the mobile device 108 may be embodied as a laptop computer, a tablet computer, a smartphone, a mobile computer, a smart device, a wearable computer system, or other computer or computer device. The mobile device 108 may include one or more processors, memory, input/output (“I/O”) subsystems, data storages, communication systems, and peripheral devices, including at least one camera 126. As suggested in FIG. 1, and further described below, the camera 126 of the mobile device 108 may be operated to capture one or more images 300 of the visual display 122 of the imaging device 102 and/or of the visual display 124 of the local device 104. In the illustrative embodiment, the mobile device 108 is not directly connected to the imaging device 102.

The links 110, 112, 114, 120 may each be embodied as any type of communication network, circuit, device, or collection thereof, capable of enabling communications between the linked devices. Each link 110, 112, 114, 120 may be configured to use any one or more communication technologies (e.g., wireless or wired communications) and associated protocols (e.g., Ethernet, Bluetooth®, Wi-Fi®, WiMAX, LTE, 5G, etc.) to effect such communication. When embodied as a network, any of the communication links 110, 112, 114, 120 may include one or more routers, switches, gateways, computers, and/or other intervening devices. By way of example, the link 112 may be embodied as or otherwise include one or more local or wide area networks, cellular networks, publicly available global networks (e.g., the Internet), an ad hoc network, a short-range communication network or link, or any combination thereof. In the illustrative embodiment, the link 110 consists of a wired connection between the imaging device 102 and the local device 104, the link 120 consists of a wired connection between the C-arm 116 and the display console 120, but the links 112, 114 include wireless connections.

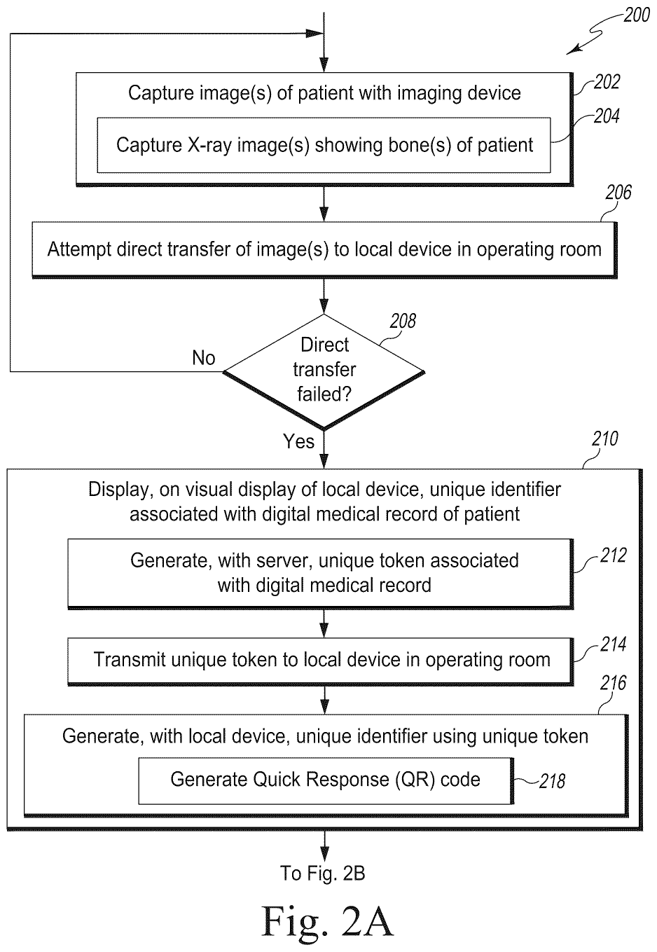

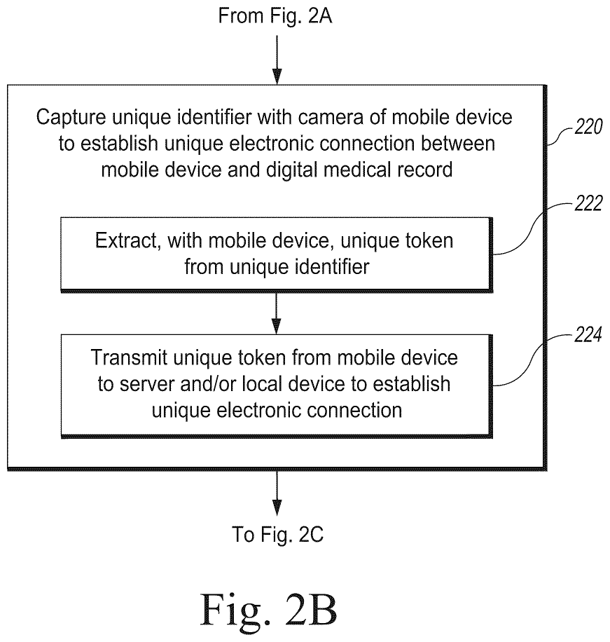

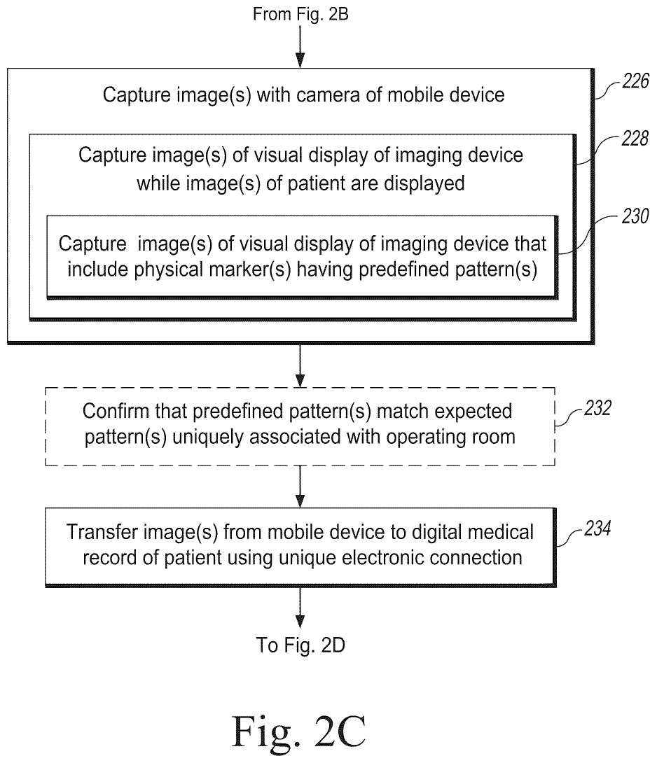

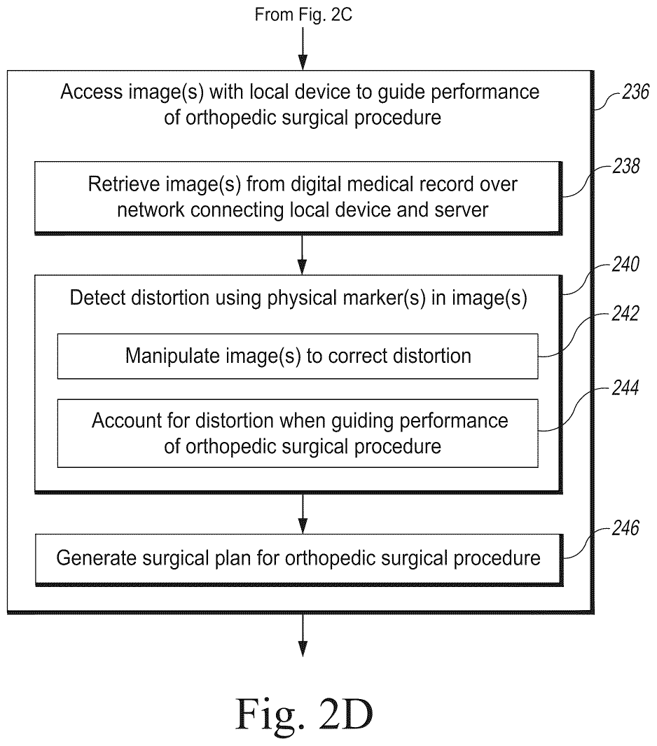

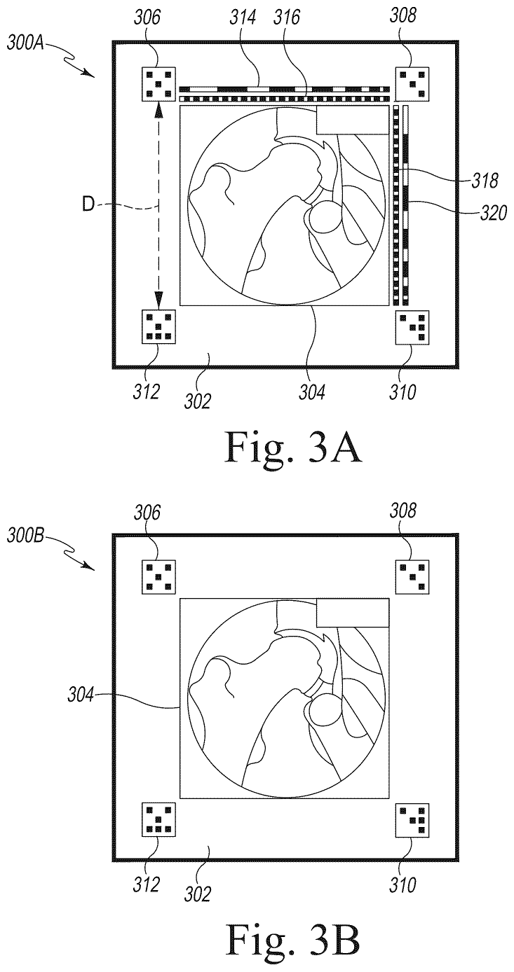

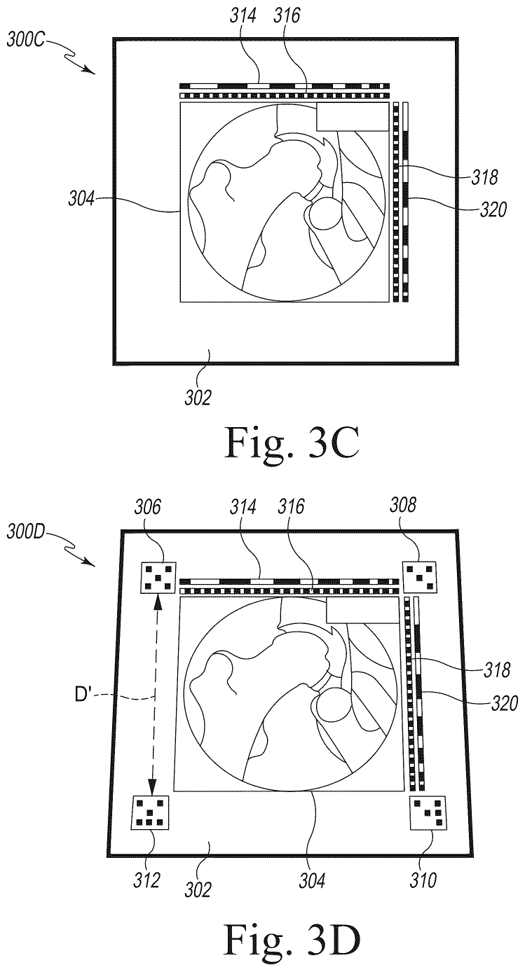

Referring now to FIGS. 2A-2D, in use, the system 100 is configured to execute a method 200 for image capture. The method 200, or portions thereof, may be embodied as a set of executable instructions stored on and executable by the system 100 or one or more portions thereof (e.g., the imaging device 102, the local device 104, the server 106, and/or the mobile device 108). The method 200 begins with block 202 in which the imaging device 102 captures one or more images 304 of the patient. In some embodiments, block 202 involves block 204 in which the imaging device 102 captures one or more X-ray images 304 showing one or more bones of the patient. One illustrative example of an X-ray image 304, showing a femur and a pelvis of a patient, which may be captured by the C-arm 116 in blocks 202, 204 is shown in each of FIGS. 3A-3D.

After blocks 202, 204, in some embodiments, the method 200 may proceed to block 206, in which the imaging device 102 attempts to transfer one or more images 304 it has captured to local device 102 for incorporation into the patient's digital medical record 128 (either by the local device 102 or after further transfer of the image(s) 304 to the server 106). To do so, the imaging device 102 may attempt to transmit one or more digital files containing the image(s) 304 to the local device 102 via the wired connection 110. In such embodiments, the method 200 next proceeds to block 208 in which the system 100 determines whether or not the attempted transfer in block 206 failed. If not (i.e., if the transfer of the image(s) 304 to the patient's digital medical record 128 was successful), the method 200 restarts (or concludes). However, if the attempted transfer in block 206 did fail, the method 200 instead continues to block 210, described below. The attempted transfer of the image(s) 304 to the patient's digital medical record 128 may fail for any number of reasons. In some situations, the wired connection 110 may fail due hardware and/or software issues. In other situations, the wired connection 110 may not be removed (or not created) due to a concern over presenting a tripping hazard in the operating room.

In block 210 of the method 200, the local device 104 displays a unique identifier 130 on the visual display 124 while the local device 104 is positioned in the operating room. The unique identifier 130 is associated with, or particularly tied to, the digital medical record 128 of the patient who is also in the operating room. The patient who is present in the operating room may be known to the local device 104 because the orthopaedic surgeon has previously logged into the software running on the local device and selected patient-specific data in that software (e.g., case information for the orthopaedic surgical procedure being performed by the orthopaedic surgeon). As shown in FIG. 1, the unique identifier 130 may be illustratively embodied as a Quick Response (QR) code that encodes data associated with the patient in the operating room.

In some embodiments (particularly those where some or all of the digital medical record 128 is maintained on the server 106), block 210 of the method 200 may involve performing blocks 212-216 to generate the unique identifier 130. In block 212, the server 106 generates a unique token associated with the digital medical record 128 of the patient. In block 214, the server 106 securely transmits that unique token over the link 112 to the local device 104. Then, in block 216, the local device 104 uses the unique token to generate the unique identifier 130, so that it can be displayed on the visual display 124. In such embodiments, the unique identifier 130 may be embodied as a QR code that encodes data specific to the unique token initially generated by the server 106.

After block 210, while the unique identifier 130 is being displayed on the visual display 124, the mobile device 108 may be operated to capture an image including the unique identifier 130 using the camera 126 of the mobile device 108. For instance, an application running on the mobile device 108 may receive image data from the camera 126 that includes the unique identifier 130. The application may recognize the unique identifier 130 and take one or more actions in response that establish a unique electronic connection between the mobile device 108 and the patient's digital medial record 128. As discussed further below, this unique electronic connection may allow the mobile device 108 to securely transfer data, including image data, to the patient's digital medial record 128, when such transfers would otherwise not be allowed by security measures on the server 106 and/or the local device 104. In some embodiment, block 220 may involve block 222, in which the mobile device 108 extracts the unique token from the unique identifier 130 (e.g., by decoding data encoded in the unique identifier 130), and block 224, in which the mobile device 108 then transmits the unique token to the server 106 to establish the unique electronic connection with the patient's digital medical record 128.

After establishing the unique electronic connection in block 220, the method 200 proceeds to block 226, in which the mobile device 108 is operated to capture one or more images 300 of the patient with the camera 126 of the mobile device 108. In the illustrative embodiment, block 226 involves block 228 in which the camera 126 is used to capture one or more images 300 of the visual display 122 of the imaging device 102 while the visual display 122 of the imaging device 102 displays the one or more initial images 304 (e.g., the X-ray image 304 showing bones of the patient). Several illustrative examples of images 300A, 300B, 300C, 300D that could be captured by the camera 136 during block 228 are shown in FIGS. 3A-3D. Each of the images 300A-D includes an X-ray image 304 of the patient that was displayed on the visual display 122 of the imaging device 102 while the image 300A-D was captured by the camera 126. Additionally, each of the images 300A-D includes a bezel 302 of the visual display 122 of the imaging device 102 surrounding the X-ray image 304.

In some embodiments, the visual display 122 of the imaging device 102 includes one or more physical markers 306-320 having a predefined pattern. By way of example, as shown in FIGS. 3A-3D, the one or more physical markers 306-320 may be arranged on the bezel 302 of the visual display 122. In such embodiments, blocks 226, 228 of the method 200 may also involve block 230 in which the camera 126 is used to capture one or more images 300 of the visual display 122 of the imaging device 102 that further include the physical marker(s) 306-320 (in addition to the image 304 of the patient, e.g., the X-ray image 304). When used, the predefined pattern of the physical marker(s) 306-320 includes at least three identifiable locations spaced apart from one another at predefined distances. The three (or more) identifiable locations may all be part of a single physical marker or may be constituted by three (or more) separate physical markers. For instance, as shown in the FIG. 3A, the physical markers 306, 312 are each independently identifiable (due to their different patterns) and are spaced apart from one another at a predefined distance D. Similarly, the physical markers 308, 310 are each independently identifiable (due to their different patterns), are spaced apart from one another at a predefined distance (not labeled), and are each spaced apart from each of the physical markers 306, 312 at different predefined distances (not labeled). In some embodiments, one or more of the physical markers 306-312 may be embodied as a QR code that encodes data about the imaging device 102 and/or a specific operating room with which the imaging device is associated.

In the illustrative embodiment of FIG. 3A, the display 122 of the imaging device 102 further includes scaling strips 314, 316, 318, 320 arranged along different sides of the bezel 302. The scaling strips 314, 316 are illustratively arranged along a horizontal portion of the bezel 302, while the scaling strips 318, 320 are illustratively arranged along a vertical portion of the bezel 302. The scaling strips 316, 318 each include alternating dark and light sections of equal width, while scaling strips 314, 320 each include alternating dark and light sections of increasing width along their lengths. The different patterns of the various physical markers 306-320 may be used by the system 100 to detect various characteristics of the images 300 captured by the camera 126 in block 230, as further discussed below.

Different embodiments may utilize subsets of the physical markers 306-320 shown in the image 300A of FIG. 3A. For instance, FIG. 3B shows an embodiment in which only the physical markers 306-312 are present on the visual display 122 and, thus, the image 300B includes only the physical markers 306-312 and not the physical markers 314-320. As another example, FIG. 3C shows an embodiment in which only the physical markers 314-320 are present on the visual display 122 and, thus, the image 300C includes only the physical markers 314-320 and not the physical markers 306-312. In some embodiments, the physical markers 306-320 (including subsets thereof) may be applied to the bezel 302 of the visual display 122 as one or more decals. To prevent tampering with the physical markers 306-320 and impede unauthorized uses of the system 100, each decal may be configured to destroy at least a portion of the predefined pattern if the decal is removed from the visual display 122 of the imaging device 102. In such embodiments, if the decal(s) are moved from the visual display 122 of the imaging device 102 to a new (unauthorized) visual display, the predefined pattern will no longer be present in images of the new (unauthorized) visual display.

After block 226, the method 200 may optionally proceed to block 232. Specifically, in embodiments of method 200 involving block 230 (in which the camera 126 captures image(s) 300 including one or more physical markers 306-320 having a predefined pattern), the mobile device 108 may detect the pattern of the one or more physical markers 306-320 in the captured image(s) 300 and then compare at least a portion of that pattern to an expected pattern that is uniquely associated with the imaging device 102 in the operating room where the application running on the mobile device 108 has been authorized to be used. If the detected pattern matches the expected pattern, the current use is authorized, and the method 200 may proceed to block 234, described below. However, if the pattern detected in the captured image(s) 300 does not match the expected pattern (e.g., because the mobile device 108 and/or the physical markers 306-320 are being used with a different imaging device 102 than the one authorized by the software vendor), the application running on the mobile device 108 identifies the current use as unauthorized and will not transmit the image(s) 300 (or otherwise proceed with the method 200). In alternative embodiments, optional block 232 may instead be performed by the server 106 and/or the local device 104 (rather than the mobile device 108) after the image has been transmitted (e.g., before the image is permitted to be added to the digital medical record or before the image can be used accessed for surgical planning).

After optional block 232 results in a successful confirmation (when used), or after block 226 (when block 232 is not used), the method 200 proceeds to block 234 in which the mobile device 108 transfers the image(s) 300 to the digital medical record 128 of the patient using the unique electronic connection established in block 220. In some embodiments, block 234 may involve wirelessly transmitting the image(s) 300 to the digital medical record 128 of the patient. For instance, the mobile device 106 may wirelessly transmit the image(s) 300 to the server 106 for incorporation into the digital medical record 128. Alternatively, where some or all of the digital medical record 128 is maintained on the local device 104, the mobile device 106 may wirelessly transmit the image(s) 300 to the local device 104 for incorporation into the digital medical record 128.

After block 234, the method 200 proceeds to block 236 in which the local device 104 accesses the image(s) 300 to guide performance of the orthopaedic surgical procedure on the patient. In some embodiments, when the digital medical record 128 of the patient is stored on the server 106, block 236 may involve block 238 in which the local device 104 retrieves the image(s) 300 from the digital medical record 128 over a network 112 connecting the local device 104 and the server 106. In other embodiment, where at least a portion of the digital medical record 128 including the image(s) 300 is maintained on the local device 104, the local device 104 may instead retrieve the image(s) 300 from local memory or data storage.

In some embodiments of the method 200, block 236 may involve block 240 in which the system 100 detects distortion in one or more images 300 using the physical markers 306-320 present in the image(s) 300. For instance, as illustrated in FIG. 3D, if the camera 126 is not properly aligned with the visual display 122 in block 226, the resulting image 300D, including the embedded image 304 of the patient's anatomy, will be distorted. Unless corrected or accounted for, this distortion may result in the surgical planning software of the system 100 using incorrect anatomical measurements, potentially impacting clinical outcomes. To detect distortion in block 240, the system 100 may detect the pattern of the physical marker(s) 306-320 in the captured image 300D (see FIG. 3D) and then compare at least a portion of that detected pattern to a predefined pattern expected for the same portion of the physical marker(s) 306-320 (see FIG. 3A, for example). Block 240 may involve determining the distance D′ between two of the physical markers 306, 312 in the image 300D. If the distance D′ in the image 300D is different form the distance D in image 300A, the image 300D may be identified as distorted. It will be appreciated that using the distances D, D′ between the physical markers 306, 312 is merely exemplary and that distortions in groupings of physical marker(s) 306-320 (and even individual markers) may be detected in block 240. In some embodiments, the portion(s) of the physical marker(s) 306-320 used for detecting distortion may be standardized across imaging devices 102 and/or operating rooms, whereas other portions of the physical marker(s) 306-320 may uniquely identify a particular imaging device 102 or operating room associated with the physical marker(s) 306-320.

When image distortion is detected in block 240, the method 200 may also involve block 242 in which the system 100 manipulates the distorted image 300D to correct the distortion. For instance, depending on the specific distortion detected, the system 100 may be able to appropriately adjust the pixels of the distorted image 300D to reconstitute it as a corrected image (similar to image 300A of FIG. 3A), using one or more known image processing algorithms. When block 242 is performed, the system 100 may then use the corrected image (rather than the distorted image 300D) to guide performance of the orthopaedic surgical procedure on the patient in block 236. Alternatively, instead of manipulating the distorted image 300D to generate a corrected image, the method 200 may involve block 244 in which the system 100 quantifies how the detected distortion has impacted the position of anatomical features of interest in the distorted image 300D and then accounts for this distortion when guiding performance of the orthopaedic surgical procedure. For instance, if the surgical planning software of the system 100 utilizes a measured distance between two anatomical features (e.g., a femoral head center and a superior point on the greater trochanter) as an input when analyzing the positioning of prosthetic components (e.g., of a hip prosthesis), and the system 100 is able to determine that distortion in the image 300D has reduced the apparent distance between these points by 10%, the system 100 can increase the relevant distance measured from the image 300D by 10% (to account for the distortion) before providing the value as an input to the surgical planning software. While the foregoing example uses simple linear distortion, it will be appreciated that the distortion may also have a non linear element (e.g., from a wide angle camera lens), and the system 100 can perform more complicated corrections to account for such distortion. It is contemplated that the detection of distortion in block 240 and the possible remedial actions in block 242, 244 may be performed by the mobile device 108, the server 106, and/or the local device 104 in various embodiments.

In the illustrative embodiment of the method 200, block 236 also involved block 246 in which the image(s) 300 are used to generate a surgical plan for the orthopaedic surgical procedure. As described above, various anatomical measurements made from the image(s) 300 may be input to a surgical planning algorithm to determine the positioning of various prosthetic components relative to the bones of the patient. The surgical plan generated in block 246 may instruct the surgeon on steps to perform to surgically prepare the patient's bones to receive the prosthetic implants in the planned position(s). Additionally or alternatively, the surgical plan generated in block 246 may be used to control a robotic surgical system that assists the surgeon in surgically preparing the patient's bones to receive the prosthetic implants in the planned position(s).

While the disclosure has been illustrated and described in detail in the drawings and foregoing description, such an illustration and description is to be considered as illustrative and not restrictive in character, it being understood that only illustrative embodiments have been shown and described and that all changes and modifications that come within the spirit of the disclosure are desired to be protected.

There are a plurality of advantages of the present disclosure arising from the various features of the methods, apparatuses, and systems described herein. It will be noted that alternative embodiments of the methods, apparatuses, and systems of the present disclosure may not include all of the features described yet still benefit from at least some of the advantages of such features. Those of ordinary skill in the art may readily devise their own implementations of the methods, apparatuses, and systems that incorporate one or more of the features of the present invention and fall within the spirit and scope of the present disclosure as defined by the appended claims.

1. A method of operating an orthopaedic surgical system, the method comprising:
displaying a unique identifier on a visual display of a first device positioned in an operating room, the unique identifier being associated with a digital medical record of a patient positioned in the operating room,
capturing the unique identifier with a camera of a second device positioned in the operating room,
establishing a unique electronic connection between the second device and the digital medical record of the patient based on the unique identifier,
capturing one or more images of the patient with the camera of the second device,
transferring the one or more images from the second device to the digital medical record of the patient using the unique electronic connection, and
accessing the one or more images with the first device to guide performance of an orthopaedic surgical procedure on the patient.

2. The method of claim 1, wherein transferring the one or more images from the second device to the digital medical record of the patient using the unique electronic connection comprises wirelessly transmitting the one or more images.

3. The method of claim 1, wherein accessing the one or more images with the first device to guide performance of the orthopaedic surgical procedure comprises generating a surgical plan for the orthopaedic surgical procedure with the first device using the one or more images.

4. The method of claim 1, wherein the digital medical record of the patient is stored on a server positioned outside of the operating room, and wherein accessing the one or more images with the first device comprises retrieving the one or more images from the digital medical record over a network connecting the first device and the server.

5. The method of claim 4, further comprising:
generating, with the server, a unique token associated with the digital medical record of the patient,
transmitting the unique token from the server to the first device, and
generating, with the first device, the unique identifier using the unique token.

6. The method of claim 5, wherein establishing the unique electronic connection between the second device and the digital medical record of the patient comprises:
extracting, with the second device, the unique token from the unique identifier, and
transmitting the unique token from the second device to the server to establish the unique electronic connection.

7. The method of claim 1, further comprising capturing one or more initial images of the patient with a third device that is not connected to the second device, wherein capturing the one or more images of the patient with the second device comprises capturing, with the camera of the second device, one or more images of a visual display of the third device while the visual display of the third device displays the one or more initial images.

8. The method of claim 7, wherein capturing the one or more initial images of the patient with the third device comprises capturing one or more X-ray images showing one or more bones of the patient.

9. The method of claim 8, wherein displaying the unique identifier on the visual display of the first device is performed in response to a failure of an attempted transfer of the one or more initial images of the patient from the third device to the digital medical record.

10. The method of claim 8, wherein the visual display of the third device includes one or more physical markers having a predefined pattern, and wherein the one or more images of the visual display of the third device captured by the second device comprise the one or more initial images of the patient and the one or more physical markers.

11. The method of claim 10, wherein the predefined pattern of the one or more physical markers comprises at least three identifiable locations spaced apart from one another at predefined distances.

12. The method of claim 11, further comprising detecting distortion in the one or more images captured by the second device by determining that the at least three identifiable locations are not spaced apart from one another at the predefined distances in the one or more images.

13. The method of claim 12, further comprising manipulating the one or more images captured by the second device to correct the distortion.

14. The method of claim 12, further comprising accounting for the distortion in the one or more images when guiding the performance of the orthopaedic surgical procedure on the patient.

15. The method of claim 10, wherein the second device confirms that the predefined pattern of the one or more physical markers in the one or more images of the visual display of the third device captured by the second device matches an expected pattern uniquely associated with the third device prior to transferring the one or more images to the digital medical record of the patient using the unique electronic connection.

16. The method of claim 15, wherein each physical marker of the one or more physical markers comprises a decal applied to the visual display of the third device, and wherein each decal is configured to destroy at least a portion of the predefined pattern if the decal is removed from the visual display of the third device.

17. A method of operating an orthopaedic surgical system, the method comprising:
capturing an X-ray image showing one or more bones of a patient using an imaging device positioned in an operating room,
displaying the X-ray image on a visual display associated with the imaging device, wherein the visual display includes one or more physical markers having a predefined pattern,
capturing a digital image of the visual display with a camera positioned in the operating room, wherein the digital image comprises the one or more physical markers and the X-ray image displayed on the visual display, and
comparing a pattern of the one or more physical markers shown in the second image to the predefined pattern expected for the one or more physical markers to identify distortion in the digital image.

18. The method of claim 17, wherein the predefined pattern of the one or more physical markers comprises at least three identifiable locations spaced apart from one another at predefined distances, and wherein comparing the pattern of the one or more physical markers shown in the digital image to the predefined pattern expected for the one or more physical markers comprises determining that the at least three identifiable locations are not spaced apart from one another at the predefined distances to identify distortion in the digital image.

19. The method of claim 18, further comprising manipulating at least a portion of the digital image corresponding to the X-ray image to correct the distortion.

20. The method of claim 18, further comprising accounting for the distortion when calculating a distance or an angle between anatomical features shown in a portion of the digital image corresponding to the X-ray image.
